(19) United States
(12) Patent Application Publication
(10) Pub. No.: US 2022/0401440 A1
(43) Pub. Date: Dec. 22, 2022

(54) DOSING REGIMENS FOR A PROTEIN KINASE C INHIBITOR

(71) Applicant: IDEAYA BIOSCIENCES, INC., South San Francisco, CA (US)

(72) Inventors: Julie Hambleton, South San Francisco, CA (US); Jianhong Wang, Foster City, CA (US)

(21) Appl. No.: 17/756,095

(22) PCT Filed: Nov. 18, 2020
(86) PCT No.: PCT/US2020/061065

§ 371 Date: May 17, 2022

Related U.S. Application Data
(60) Provisional application No. 62/936,993, filed on Nov. 18, 2019.
(60) Provisional application No. 62/988,483, filed on Mar. 12, 2020.

(51) Int. Cl. A61K 31/497; A61K 45/06

(52) U.S. Cl. CPC A61K 31/497; A61K 45/06

(57) ABSTRACT

Disclosed herein are dosing regimens for the treatment of cancer mediated by protein kinase C (PKC) with 3-amino-N-(3-(4-amino-4-methylpiperidin-1-yl)pyridin-2-yl)-6-(3-(trifluoromethyl)pyridin-2-yl)pyrazine-2-carboxamide, Compound (I), or a pharmaceutically acceptable salt thereof.

### RELATED APPLICATIONS

This application claims priority to U.S. Provisional Application No. 62/936,993, filed Nov. 18, 2019 and U.S. Provisional Application No. 62/988,483, filed Mar. 12, 2020. The entire contents of both applications are hereby incorporated by reference in their entireties.

### FIELD

Disclosed herein are dosing regimens for the treatment of cancer mediated by protein kinase C (PKC) with 3-amino-N-(3-(4-amino-4-methylpiperidin-1-yl)pyridin-2-yl)-6-(3-(trifluoromethyl)pyridin-2-yl)pyrazine-2-carboxamide having the structure:

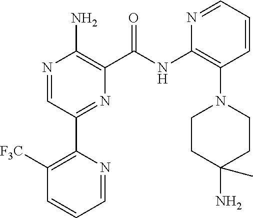 [SMILES: CC1(N)CCN(c2cccnc2NC(=O)c2nc(-c3ncccc3C(F)(F)F)cnc2N)CC1]

(herein referred to as Compound (I)), or a pharmaceutically acceptable salt thereof.

### BACKGROUND

Compound (I) is a selective PKC inhibitor and is disclosed in Example 9 of the PCT application publication No. WO 2016/020864, filed on Aug. 5, 2015 for use in the treatment of diseases or disorders mediated by PKC.

PKC belongs to a family of closely related protein kinases that are involved in various aspects of signal transduction, such as transmitting extracellular growth factor or cytokine signals to other protein kinases involved in cellular proliferation or transcription regulation. PKC is important for signal transduction and survival of cells with constitutively active mutations in GNAQ or GNA11. Activating mutations in GNAQ or GNA11 are found in approximately 90a of uveal melanoma patients, resulting in a dependency on PKC activity, which sensitize these tumors to the effects of Compound (I).

Patients with metastatic uveal melanoma have a very poor prognosis, and there are no FDA-approved therapies for this disease. Without treatment, median overall survival of patients with metastatic uveal melanoma is approximately ten months. Historical response rates for uveal melanoma generally range from 0% to 10% across treatment types. The poor prognosis associated with metastatic disease and the lack of effective therapies highlight the need for novel therapeutic approaches that specifically target metastatic uveal melanoma. Compound (I) is currently in clinical trials for treatment of metastatic uveal melanoma (MUM) and GNAQ/11 mutated tumors. As of September 2018, an ongoing Phase 1 clinical trial conducted by Novartis (ClinicalTrials.gov Identifier: NCT02601378) enrolled 68 patients in a dose escalation monotherapy arm, with 38 patients receiving Compound (I) once a day (QD) and 30 patients receiving Compound (I) twice a day (BID). In preliminary findings from 68 patients as of September 2018, Novartis reported a total of six confirmed partial responses (PRs) and two unconfirmed PRs among the 45 patients that exhibited stable disease (SD). Four of these confirmed PRs and 18 of the patients with SD (2 being unconfirmed PRs) as their best response belong to a cohort of patients that received BID dosing of 200 to 400 mg of Compound (I). Dose limiting toxicities (DLTs) were reported in 7 of 38 patients on the QD schedule and in 2 of 17 patients on the BID schedule who were evaluable for the Bayesian logistic regression model. The most common DLT was hypotension, which was manageable and resolved quickly with intravenous fluids, dose interruption, and/or dose reduction. In the dose escalation study of the Novartis clinical trial, maximum tolerated doses were determined at 500 mg QD and 400 mg BID and the recommended dose for expansion (RDE) was declared at 300 mg BID.

For a drug to be suitable for use as a therapeutic agent, it should be administrable at dosing regimens that maximize its therapeutic efficacy while minimizing adverse effects. The present disclosure fulfills this and related needs.

### SUMMARY

Disclosed herein are dosing regimens for the treatment of cancer mediated by PKC with Compound (I), or a pharmaceutically acceptable salt thereof. The methods of treatment disclosed herein are based, at least in part, on findings that patients who were administered 200 mg BID of Compound (I) for the first 7 days, followed by 400 mg BID of Compound (I) for the remainder of a 28-day first dosing cycle as monotherapy had lower mean exposure at C1D8 compared to C1D1 of patients who received 300 mg BID of Compound (I). Additionally, patients who were administered 200 mg BID of Compound (I) for the first 7 days, followed by 400 mg BID of Compound (I) had higher mean steady state exposure of Compound (I) vis-d-vis patients who were administered 300 mg BID (RDE) of Compound (I) for the same duration. Preliminary exposure-safety analysis suggests that free exposure, especially on day 1, is generally higher in patients who experience hypotension (see M. Roy et al., Pharmacokinetic characterization and preliminary exposure-safety/response analysis of a novel PKC inhibitor LXS196 in a phase I study in metastatic Uveal Melanoma patients, ASCPT 2019). Because the therapeutic efficacy of Compound (I) may be driven by area under the curve (AUC), dosing patients with 400 mg BID of Compound (I) with a 200 mg BID run-in dose is an improved dosing regimen for Compound (I) as it potentially minimizes the risk of hypotension while improving the therapeutic potential of Compound (I).

Accordingly, among the various aspects of the present disclosure may be noted the provision of a dosing regimen for the treatment of cancers mediated by PKC with Compound (I), or a pharmaceutically acceptable salt thereof.

In a first aspect, provided is a method of treating cancer mediated by PKC comprising administering to a patient in need thereof, a therapeutically effective amount of 3-amino-N-(3-(4-amino-4-methylpiperidin-1-yl)pyridin-2-yl)-6-(3-(trifluoromethyl)pyridin-2-yl)pyrazine-2-carboxamide (Compound (I)), or a pharmaceutically acceptable salt thereof, as a monotherapy, in a dosing regimen comprising a first dosing cycle comprising a first dosing series followed by a second dosing series, wherein:

(a) the first dosing series comprises a dose of about 200 mg BID of compound (I), or a pharmaceutically acceptable salt thereof, and

(b) the second dosing series comprises a dose of about 400 mg BID of compound (I), or a pharmaceutically acceptable salt thereof.

In a second aspect, provided is a method of treating cancer mediated by PKC comprising administering to a patient in need thereof, a therapeutically effective amount of a pharmaceutical composition comprising 3-amino-N-(3-(4-amino-4-methylpiperidin-1-yl)pyridin-2-yl-6-(3-(trifluoromethyl)pyridin-2-yl)pyrazine-2-carboxamide (Compound (I)), or a pharmaceutically acceptable salt thereof as a monotherapy, in a dosing regimen comprising a first dosing cycle comprising a first dosing series followed by a second dosing series, wherein:

(a) the first dosing series comprises a dose of about 200 mg BID of compound (I), or a pharmaceutically acceptable salt thereof, and

(b) the second dosing series comprises a dose of about 400 mg BID of compound (I), or a pharmaceutically acceptable salt thereof.

Additional PKC inhibitors that can be used in above dosing regimen are compounds of Formula (I), (II), (III) and specific compounds disclosed in PCT application publication No. WO 2016/020864, the disclosure of which is incorporated herein in its entirety.

### BRIEF DESCRIPTION OF FIGURES

FIG. 1 illustrates mean plasma concentration vs. time following a single dose of 300 mg BID of Compound (I) on Cycle 1, Day 1 (n=12) and 400 mg BID of Compound (I) on Cycle 1, Day 8 (n=24). (MUM=metastatic uveal melanoma)

FIG. 2 illustrates mean steady state plasma concentration vs. time of Compound (I) for 300 mg BID and 400 mg BID dosing on Cycle 1, Day 15 and Cycle 1, Day 22 respectively. (MUM=metastatic uveal melanoma)

### DETAILED DESCRIPTION

Unless otherwise indicated, the following terms are intended to have the meaning set forth below. Other terms are defined elsewhere throughout the specification.

The use of the articles “a”, “an”, and “the” in both the specification and claims are to be construed to cover both the singular and the plural, unless otherwise indicated herein or clearly contracted the context. The terms “comprising”, “having”, “including” and “containing” are to be construed as open terms (i.e., meaning “including but not limited to”) unless otherwise noted. Additionally, whenever “comprising” or other open-ended term is used in an embodiment or claim, it is to be understood that the same embodiment or claim can be more narrowly claimed using the intermediate term “consisting essentially of” or the closed term “consisting o”.

The phrases “cancer mediated by protein kinase C” and “cancer mediated by PKC” refers to a cancer in which protein kinase C plays a role in the pathogenesis of the cancer.

The phrase “pharmaceutically acceptable salts” refers to nontoxic acid or alkaline earth metal salts of a compound of the disclosure e.g., Compound (I). These salts can be prepared in situ during the final isolation and purification of compound of present disclosure by separately reacting the base or acid functions in the compound with a suitable organic or inorganic acid or base, respectively. Representative salts include, but are not limited to, acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-naphthalene-sulfonate, oxalate, pamoate, pectinate, persulfate, 3-phenylproionate, picrate, pivalate, propionate, succinate, sulfate, tartrate, thiocyanate, p-toluenesulfonate and undecanoate. Also, the basic nitrogen-containing groups can be quaternized with such agents as alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others.

Examples of acids which may be employed to form pharmaceutically acceptable acid addition salts include such inorganic acids as hydrochloric acid, sulfuric acid and phosphoric acid and such organic acids as oxalic acid, maleic acid, methanesulfonic acid, succinic acid and citric acid. Basic addition salts can be prepared in situ during the final isolation and purification of compound of the disclosure by reacting carboxylic acid moieties with a suitable base such as the hydroxide, carbonate or bicarbonate of a pharmaceutically acceptable metal cation or with ammonia, or an organic primary, secondary or tertiary amine. Pharmaceutically acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethyl-amine, trimethylamine, triethylamine, ethylamine, and the like. Other representative organic amines useful for the formation of base addition salts include diethylamine, ethylenediamine, ethanolamine, diethanolamine, piperazine and the like.

“Disease,” as used herein, is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration and/or quality of life.

As used herein, the term “GNAQ” refers to Guanine Nucleotide-Binding Protein Alpha-Q gene that encodes the Gq alpha subunit (Gαq) and the term “GNA11” refers to Guanine Nucleotide-Binding Protein Alpha 11 genes that encodes the G11alpha subunit (Gα11) subunit.

As used herein, “mutations” can refer to changes in a polynucleotide sequence that result in changes to protein activity. Mutations can be nucleotide substitutions, such as single nucleotide substitutions, insertions, or deletions. GNAQ and GNA11 mutations are typically activating mutations, i.e., mutations that activate the PKC pathway, due to constitutive activation of the α subunit. Without being bound to a theory, it is believed that the constitutive activity results from a lack of the GTP-hydrolase activity in the mutant GNAQ or GNA11 protein. Activating mutations can also refer to mutations that result in a loss or decrease of GTP hydrolyzing activity of a Gα subunit. Mutations in GNAQ and GNA11 include a substitution of arginine in codon R183 or substitution of glutamine in codon Q209, or may be other mutations. In an embodiments, mutations in GNAQ and/or GNA11 can be selected from group comprising of: Q209P, Q209L, Q209H, Q209K, Q209Y, Q209R, Q209H, R183Q, R183, for example, GNAQ Q209 may be mutated to either P or L as well as to R or H; GNAQ R183 may be mutated to Q; GNA11 Q209 may be mutated to L as well as to P or K; GNAQ R183 may mutate to C or H. GNA11 Q209 can be mutated to L as well as rarely to P or K; also GNAQ R183 is most often mutate to C and more rarely to H.

The term “in need of treatment,” as used herein, refers to a judgment made by a physician or other caregiver that a subject requires or will benefit from treatment. This judgment is made based on a variety of factors that are in the realm of the physician's or caregivers expertise.

The terms “administration”, “administer” and the like, as they apply to, for example, to a patient refer to contact of, for example, compound (I), a pharmaceutical composition comprising same, with the patient.

The terms “treat,” “treating” or “treatment,” as used herein, refers to methods of alleviating, abating or ameliorating a disease, e.g., uveal melanoma, or condition or symptoms, preventing additional symptoms, ameliorating or preventing the underlying metabolic causes of symptoms, inhibiting the disease or condition, arresting or reducing the development of the disease or condition, relieving the disease or condition, causing regression of the disease or condition, relieving a condition caused by the disease or condition, or stopping the symptoms of the disease or condition either prophylactically and/or therapeutically.

The terms “effective amount” or “therapeutically effective amount,” as used herein, refer to an amount of a compound described herein e.g., Compound (I) or a pharmaceutical composition comprising a compound described herein, being administered which will treat the disease or condition being treated. An appropriate “effective” amount in any individual case may be determined using techniques, such as a dose escalation study. In connection with the administration of the drug, an “effective amount” indicates an amount that results in a beneficial effect for patients, such as an improvement of symptoms, a cure, a reduction in disease load, reduction in tumor mass or cell numbers, extension of life, improvement in quality of life, or other effect generally recognized as positive by medical doctors familiar with treating the particular type of disease or condition.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

The terms “patient” or “subject” refers to a mammal, preferably human.

Unless otherwise specified, the weight or dosage referred to herein for a particular compound (e.g., Compound (I)) of the disclosure is the weight or dosage of the compound itself, not that of a salt thereof, which can be different to achieve the intended therapeutic effect. For example, the weight or dosage of a corresponding salt of Compound (I) suitable for the methods, compositions, or combinations disclosed herein may be calculated based on the ratio of the molecular weights of the particular salt of Compound (I) and Compound (I) itself.

The terms “about,” “approximately,” or “approximate,” when used in connection with a numerical value, means that a collection or range of values is included. As used herein “about X” includes a range of values that are ±25%, ±20%, ±10%, ±5%, ±2%, ±1%, ±0.5%, ±0.2%, or ±0.1% of X, where X is a numerical value. In one embodiment, the term “about” refers to a range of values which are 25% more or less than the specified value. In another embodiment, the term “about” refers to a range of values which are 20% more or less than the specified value. In yet another embodiment, the term “about” refers to a range of values which are 10% more or less than the specified value. Preferably, the term “about” refers to a range of values which are 5% more or less than the specified value. More preferably, the term “about” refers to a range of values which are 1% more or less than the specified value.

The term “BID” means twice a day.

### EMBODIMENTS

In embodiments 1 to 70 below, the present disclosure includes:

### Embodiment 1

In Embodiment 1, provided is a method of treating cancer mediated by PKC comprising administering to a patient in need thereof, a therapeutically effective amount of 3-amino-N-(3-(4-amino-4-methylpiperidin-1-yl)pyridin-2-yl)-6-(3-(trifluoromethyl)pyridin-2-yl)pyrazine-2-carboxamide (Compound (I)), or a pharmaceutically acceptable salt thereof, as a monotherapy, in a dosing regimen comprising a first dosing cycle comprising a first dosing series followed by a second dosing series, wherein:

(a) the first dosing series comprises a dose of about 200 mg BID of compound (I), or a pharmaceutically acceptable salt thereof, and

(b) the second dosing series comprises a dose of about 400 mg BID of compound (I), or a pharmaceutically acceptable salt thereof.

### Embodiment 2

In Embodiment 2, provided is a method of treating cancer mediated by PKC comprising administering to a patient in need thereof, a therapeutically effective amount of a pharmaceutical composition comprising 3-amino-N-(3-(4-amino-4-methylpiperidin-1-yl)pyridin-2-yl)-6-(3-(trifluoromethyl)pyridin-2-yl)pyrazine-2-carboxamide (Compound (I)), or a pharmaceutically acceptable salt thereof, and at least one pharmaceutically acceptable excipient, as a monotherapy, in a dosing regimen comprising a first dosing cycle comprising a first dosing series followed by a second dosing series, wherein:

(a) the first dosing series comprises a dose of about 200 mg BID of compound (I), or a pharmaceutically acceptable salt thereof, and

(b) the second dosing series comprises a dose of about 400 mg BID of compound (I), or a pharmaceutically acceptable salt thereof.

### Embodiment 3

In Embodiment 3, the method of embodiment I or 2 is wherein the length of the first dosing series is 5 to 10 days.

### Embodiment 4

In Embodiment 4, the method of embodiment 3 is wherein the length of the second dosing series is 18 to 23 days provided the length of first dosing cycle comprising first dosing series and second dosing series is 28 days, e.g., the length of the second dosing series is 23 days when the length of first dosing series is 5 days.

### Embodiment 5

In Embodiment 5, the method of any one of embodiments 1 to 4 is wherein the first dosing cycle comprises one first dosing series, and compound (I) is administered on days 1 to consecutively of the first dosing series of the first dosing cycle.

### Embodiment 6

In Embodiment 6, the method of any one of embodiments 1 to 4 is wherein the first dosing cycle comprises one first dosing series, and compound (I) is administered on days 1 to 6 consecutively of the first dosing series of the first dosing cycle.

### Embodiment 7

In Embodiment 7, the method of any one of embodiments 1 to 4 is wherein the first dosing cycle comprises one first dosing series, and compound (I) is administered on days 1 to 7 consecutively of the first dosing series of the first dosing cycle.

### Embodiment 8

In Embodiment 8, the method of embodiment 5 is wherein compound (I) is administered on days 1 to 23 consecutively of the second dosing series or days 6 to 28 of the first dosing cycle.

### Embodiment 9

In Embodiment 9, the method of embodiment 6 is wherein compound (I) is administered on days 1 to 22 consecutively of the second dosing series or days 7 to 28 of the first dosing cycle.

### Embodiment 10

In Embodiment 10, the method of embodiment 7 is wherein compound (I) is administered on days 1 to 21 consecutively of the second dosing series or days 8 to 28 of the first dosing cycle.

### Embodiment 11

In Embodiment 11, the method of any one of embodiments 1 to 10 is wherein the dosing regimen comprises one or more additional dosing cycles of second dosing series wherein each additional dosing cycle is 28 days.

### Embodiment 12

In Embodiment 12, the method of embodiment 11 is wherein compound (I) is administered consecutively for 28 days of each additional dosing cycle.

### Embodiment 13

In Embodiment 13, the method of embodiment I 1 or 12 is wherein the number of additional dosing cycles of the second dosing series is at least 2.

### Embodiment 14

In Embodiment 14, the method of embodiment 11 or 12 is wherein the number of additional dosing cycles of the second dosing series is at least 3.

### Embodiment 15

In Embodiment 15, the method of embodiment 11 or 12 is wherein the number of additional dosing cycles of the second dosing series is at least 4.

### Embodiment 16

In Embodiment 16, the method of embodiment I 1 or 12 is wherein the number of additional dosing cycles of the second dosing series is at least 5.

### Embodiment 17

In Embodiment 17, the method of embodiment 11 or 12 is wherein the number of additional dosing cycles of the second dosing series is at least 6.

### Embodiment 18

In Embodiment 18, the method of embodiment 11 or 12 is wherein the number of additional dosing cycles of the second dosing series is at least 7.

### Embodiment 19

In Embodiment 19, the method of embodiment 11 or 12 is wherein the number of additional dosing cycles of the second dosing series is at least 8.

### Embodiment 20

In Embodiment 20, the method of embodiment I 1 or 12 is wherein the number of additional dosing cycles of the second dosing series is at least 10.

### Embodiment 21

In Embodiment 21, the method of embodiment 11 or 12 is wherein the number of additional dosing cycles of the second dosing series is at least 12.

### Embodiment 22

In Embodiment 22, the method of embodiment 11 or 12 is wherein the number of additional dosing cycles of the second dosing series is at least 16.

### Embodiment 23

In Embodiment 23, the method of embodiment I 1 or 12 is wherein the number of additional dosing cycles of the second dosing series is at least 24.

### Embodiment 24

In Embodiment 24, the method of any one of embodiments 1 to 23 is wherein the patient is administered 150 mg BID of compound (I) in the first dosing series.

### Embodiment 25

In Embodiment 25, the method of any one of embodiments 1 to 23 is wherein the patient is administered 160 mg BID of compound (I) in the first dosing series.

### Embodiment 26

In Embodiment 26, the method of any one of embodiments 1 to 23 is wherein the patient is administered 170 mg BID of compound (I) in the first dosing series.

### Embodiment 27

In Embodiment 27, the method of any one of embodiments 1 to 23 is wherein the patient is administered 175 mg BID of compound (I) in the first dosing series.

### Embodiment 28

In Embodiment 28, the method of any one of embodiments 1 to 23 is wherein the patient is administered 180 mg BID of compound (I) in the first dosing series.

### Embodiment 29

In Embodiment 29, the method of any one of embodiments 1 to 23 is wherein the patient is administered 185 mg BID of compound (I) in the first dosing series.

### Embodiment 30

In Embodiment 30, the method of any one of embodiments 1 to 23 is wherein the patient is administered 190 mg BID of compound (I) in the first dosing series.

### Embodiment 31

In Embodiment 31, the method of any one of embodiments 1 to 23 is wherein the patient is administered 195 mg BID of compound (I) in the first dosing series.

### Embodiment 32

In Embodiment 32, the method of any one of embodiments 1 to 23 is wherein the patient is administered 200 mg BID of compound (I) in the first dosing series.

### Embodiment 33

In Embodiment 33, the method of any one of embodiments 1 to 23 is wherein the patient is administered 205 mg BID of compound (I) in the first dosing series.

### Embodiment 34

In Embodiment 34, the method of any one of embodiments 1 to 23 is wherein the patient is administered 210 mg BID of compound (I) in the first dosing series.

### Embodiment 35

In Embodiment 35, the method of any one of embodiments 1 to 23 is wherein the patient is administered 215 mg BID of compound (I) in the first dosing series.

### Embodiment 36

In Embodiment 36, the method of any one of embodiments 1 to 23 is wherein the patient is administered 220 mg BID of compound (I) in the first dosing series.

### Embodiment 37

In Embodiment 37, the method of any one of embodiments 1 to 23 is wherein the patient is administered 225 mg BID of compound (I) in the first dosing series.

### Embodiment 38

In Embodiment 38, the method of any one of embodiments 1 to 23 is wherein the patient is administered 230 mg BID of compound (I) in the first dosing series.

### Embodiment 39

In Embodiment 39, the method of any one of embodiments 1 to 23 is wherein the patient is administered 235 mg BID of compound (I) in the first dosing series.

### Embodiment 40

In Embodiment 40, the method of any one of embodiments 1 to 23 is wherein the patient is administered 240 mg BID of compound (I) in the first dosing series.

### Embodiment 41

In Embodiment 41, the method of any one of embodiments 1 to 23 is wherein the patient is administered 245 mg BID of compound (I) in the first dosing series.

### Embodiment 42

In Embodiment 42, the method of any one of embodiments 1 to 23 is wherein the patient is administered 250 mg BID of compound (I) in the first dosing series.

### Embodiment 43

In Embodiment 43, the method of any one of embodiments 1 to 42 is wherein the patient is administered 400 mg BID of compound (I) in each of the second dosing series.

### Embodiment 44

In Embodiment 47, the method of any one of embodiments 1 to 42 is wherein the patient is administered 350 mg BID of compound (I) in each of the second dosing series.

### Embodiment 45

In Embodiment 45, the method of any one of embodiments 1 to 42 is wherein the patient is administered 360 mg BID of compound (I) in each of the second dosing series.

### Embodiment 46

In Embodiment 46, the method of any one of embodiments 1 to 42 is wherein the patient is administered 370 mg BID of compound (I) in each of the second dosing series.

### Embodiment 47

In Embodiment 47, the method of any one of embodiments 1 to 42 is wherein the patient is administered 380 mg BID of compound (I) in each of the second dosing series.

### Embodiment 48

In Embodiment 48, the method of any one of embodiments 1 to 42 is wherein the patient is administered 390 mg BID of compound (I) in each of the second dosing series.

### Embodiment 49

In Embodiment 49, the method of any one of embodiments 1 to 42 is wherein the patient is administered 410 mg BID of compound (I) in each of the second dosing series.

### Embodiment 50

In Embodiment 50, the method of any one of embodiments 1 to 42 is wherein the patient is administered 420 mg BID of compound (I) in each of the second dosing series.

### Embodiment 51

In Embodiment 51, the method of any one of embodiments 1 to 42 is wherein the patient is administered 430 mg BID of compound (I) in each of the second dosing series.

### Embodiment 52

In Embodiment 52, the method of any one of embodiments 1 to 42 is wherein the patient is administered 440 mg BID of compound (I) in each of the second dosing series.

### Embodiment 53

In Embodiment 53, the method of any one of embodiments 1 to 42 is wherein the patient is administered 450 mg BID of compound (I) in each of the second dosing series.

### Embodiment 54

In Embodiment 54, the method of any one of embodiments 1 to 42 is wherein the dosing within each cycle is continuous.

### Embodiment 55

In Embodiment 55, the method of any one of embodiments 1 to 54 is wherein the cancer is selected from the group consisting of cutaneous melanoma, uveal melanoma, lymphoma, diffuse large B-cell lymphoma (DLBCL), ibrutinib resistant cancers, pancreatic cancer, colorectal cancer, lung adenocarcinoma, stomach cancer, cervical cancer, uterine cancer, bladder cancer, hepatocellular carcinoma, prostate cancer, breast cancer, head and neck cancer, and glioblastoma.

### Embodiment 56

In Embodiment 56, the method of any one of embodiments 1 to 55 is wherein the cancer is uveal melanoma.

### Embodiment 57

In Embodiment 57, the method of any one of embodiments 1 to 54 is wherein the cancer is metastatic uveal melanoma. In another embodiment, the cancer is nonmetastatic uveal melanoma. In yet another embodiment, the cancer is not nonmetastatic uveal melanoma.

### Embodiment 58

In Embodiment 58, the method of any one of embodiments 1 to 54 is wherein the cancer is selected from the group consisting of pancreatic cancer, colorectal cancer, lung adenocarcinoma, cutaneous melanoma, stomach cancer, cervical cancer, uterine cancer, bladder cancer, hepatocellular carcinoma, prostate cancer, breast cancer, head and neck cancer, and glioblastoma.

### Embodiment 59

In Embodiment 59, the method of any one of embodiments 1 to 54 and 56 to 58 is wherein the patient in need thereof harbors one or more GNAQ or GNA11 mutation.

### Embodiment 60

In Embodiment 60, the method of embodiment 55 is wherein the cancer is diffuse large B-cell lymphoma (DLBCL).

### Embodiment 61

In Embodiment 61, the method of embodiment 60 is wherein diffuse large B-cell lymphoma (DLBCL) is treated in a patient harboring CD79 mutations.

### Embodiment 62

In Embodiment 62, the method of any one of embodiments 1 and 3 to 60 is wherein Compound (I) is administered as powder in a capsule.

### Embodiment 63

In Embodiment 63, the method of any one of embodiments 2 to 60 is wherein Compound (I) is administered as a tablet formulation.

### Embodiment 64

In Embodiment 64, the method of embodiment 59 is wherein the patient in need thereof harbors a GNAQ mutation.

### Embodiment 65

In Embodiment 65, the method of embodiment 64 is wherein the GNAQ mutation is one of Q209P, Q209L, Q209H, Q209K, or Q209Y.

### Embodiment 66

In Embodiment 66, the method of embodiment 59 is wherein the patient in need thereof harbors a GNA11 mutation.

### Embodiment 67

In Embodiment 67, the method of embodiment 66 is wherein the GNA11 mutation is one of Q209P, Q209L, Q209K or Q209H.

### Embodiment 68

In Embodiment 68, the method of embodiment 65 is wherein the mutation is Q209L.

### Embodiment 69

In Embodiment 68, the method of embodiment 59 is wherein the GNAQ or GNA11 mutation is the substitution of arginine in codon R183.

### Embodiment 70

In Embodiment 70, the method of embodiment 69 is wherein the GNAQ mutation is R183Q.

### Additional Aspects

In another aspect, provided herein is a method of treating cancer mediated by PKC comprising administering to a patient in need thereof, a therapeutically effective amount of 3-amino-N-(3-(4-amino-4-methylpiperidin-1-yl)pyridin-2-yl)-6-(3-(trifluoromethyl)pyridin-2-yl)pyrazine-2-carboxamide (Compound (I)), or a pharmaceutically acceptable salt thereof, as a monotherapy, in a dosing regimen comprising a first dosing cycle comprising a first dosing series followed by a second dosing series, wherein:

(a) the first dosing series comprises a dose of 180-220 mg BID of compound (I), or a pharmaceutically acceptable salt thereof, and

(b) the second dosing series comprises a dose of 360-440 mg BID of compound (I), or a pharmaceutically acceptable salt thereof.

In still another aspect, provided herein is a method of treating cancer mediated by PKC comprising administering to a patient in need thereof, a therapeutically effective amount of 3-amino-N-(3-(4-amino-4-methylpiperidin-1-yl)pyridin-2-yl)-6-(3-(trifluoromethyl)pyridin-2-yl)pyrazine-2-carboxamide (Compound (I)), or a pharmaceutically acceptable salt thereof, as a monotherapy, in a dosing regimen comprising a first dosing cycle comprising a first dosing series followed by a second dosing series, wherein:

(a) the first dosing series comprises a dose of 186-214 mg BID of compound (I), or a pharmaceutically acceptable salt thereof, and

(b) the second dosing series comprises a dose of 372-428 mg BID of compound (I), or a pharmaceutically acceptable salt thereof.

In yet another aspect, provided herein is a method of treating cancer mediated by PKC comprising administering to a patient in need thereof, a therapeutically effective amount of 3-amino-N-(3-(4-amino-4-methylpiperidin-1-yl)pyridin-2-yl)-6-(3-(trifluoromethyl)pyridin-2-yl)pyrazine-2-carboxamide (Compound (I)), or a pharmaceutically acceptable salt thereof, as a monotherapy, in a dosing regimen comprising a first dosing cycle comprising a first dosing series followed by a second dosing series, wherein:

(a) the first dosing series comprises a dose of 190-210 mg BID of compound (I), or a pharmaceutically acceptable salt thereof, and

(b) the second dosing series comprises a dose of 380-420 mg BID of compound (I), or a pharmaceutically acceptable salt thereof.

In another aspect, provided herein is a method of treating cancer mediated by PKC comprising administering to a patient in need thereof, a therapeutically effective amount of 3-amino-N-(3-(4-amino-4-methylpiperidin-1-yl)pyridin-2-yl)-6-(3-(trifluoromethyl)pyridin-2-yl)pyrazine-2-carboxamide (Compound (I)), or a pharmaceutically acceptable salt thereof, as a monotherapy, in a dosing regimen comprising a first dosing cycle comprising a first dosing series followed by a second dosing series, wherein:

(a) the first dosing series comprises a dose of 196-204 mg BID of compound (I), or a pharmaceutically acceptable salt thereof, and

(b) the second dosing series comprises a dose of 392-408 mg BID of compound (I), or a pharmaceutically acceptable salt thereof.

### Pharmaceutical Composition

Compounds disclosed herein, including Compound (I), or a pharmaceutically acceptable salt thereof (also referred to herein as “active agent”), are useful in inhibiting the growth of cancer cells. They may be used alone or in compositions together with a pharmaceutically acceptable carrier or excipient. Suitable pharmaceutically acceptable carriers or excipients include, for example, processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinyl pyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), incorporated herein by reference.

The amount of active agent that may be combined with the carrier materials to produce a single dosage form will vary depending upon the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy.

For purposes of this disclosure, a therapeutically effective dose will generally be a total daily dose administered to a host in single or divided doses may be in amounts, for example, of from 0.001 to 1000 mg/kg body weight daily and more preferred from 1.0 to 30 mg/kg body weight daily. Dosage unit compositions may contain such amounts of submultiples thereof to make up the daily dose.

Active agent may be administered orally or parenterally. The term parenteral, as used herein, includes subcutaneous injections, intravenous, intramuscular, intrasternal injection, or infusion techniques.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-propanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid find use in the preparation of injectables.

Solid dosage forms for oral administration may include capsules, tablets, pills, powders, and granules. In such solid dosage forms, the active agent may be admixed with at least one inert diluent such as sucrose lactose or starch. Such dosage forms may also comprise, as is normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

The active agent can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq. (1976).

While an active agent can be administered as the sole active pharmaceutical agent, they can also be used in combination with one or more other agents used in the treatment of cancer. The compounds of the present disclosure are also useful in combination with known therapeutic agents and anti-cancer agents, and combinations of the presently disclosed compounds with other anti-cancer or chemotherapeutic agents are within the scope of the invention. Examples of such agents can be found in Cancer Principles and Practice of Oncology, V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved.

The references cited throughout the application are incorporated herein by reference in their entirety.

### Combination Therapy

Compound (I), or a pharmaceutically acceptable salt thereof, can be administered in combination with one or more additional therapeutic agents (e.g., chemotherapeutic agents) or other prophylactic or therapeutic modalities (e.g., radiation). In such combination therapy, the various active agents frequently have different, complementary mechanisms of action. Such combination therapy may allow for a dose reduction of one or more of the agents, thereby reducing or eliminating the adverse effects associated with one or more of the agents. Furthermore, such combination therapy may have a synergistic therapeutic or prophylactic effect on the underlying disease, disorder, or condition.

As used herein, “combination” is meant to include therapies that can be administered separately, for example, formulated separately for separate administration and therapies that can be administered together in a single formulation (i.e., a “co-formulation”).

In certain embodiments, Compound (I) and a pharmaceutically acceptable salt thereof is administered or applied sequentially, e.g., where one agent is administered prior to one or more other agents. In other embodiments, Compound (I) and a pharmaceutically acceptable salt thereof is administered simultaneously, e.g., where two or more agents are administered at or a PKC inhibitor of the present invention about the same time; the two or more agents may be present in two or more separate formulations or combined into a single formulation (i.e., a co-formulation). Regardless of whether the two or more agents are administered sequentially or simultaneously, they are considered to be administered in combination for purposes of the present invention.

Compound (I), or a pharmaceutically acceptable salt thereof, may be used in combination with at least one other (active) agent in any manner appropriate under the circumstances. In one embodiment, treatment with the at least one active agent and at least Compound (I), or a pharmaceutically acceptable salt thereof, is maintained over a period of time. In another embodiment, treatment with the at least one active agent is reduced or discontinued (e.g., when the subject is stable), while treatment with Compound (I), or a pharmaceutically acceptable salt thereof, is maintained at a constant dosing regimen. In a further embodiment, treatment with the at least one active agent is reduced or discontinued (e.g., when the subject is stable), while treatment with Compound (I), or a pharmaceutically acceptable salt thereof, is reduced (e.g., lower dose, less frequent dosing or shorter treatment regimen). In yet another embodiment, treatment with the at least one active agent is reduced or discontinued (e.g., when the subject is stable), and treatment with Compound (I), or a pharmaceutically acceptable salt thereof, is increased (e.g., higher dose, more frequent dosing or longer treatment regimen). In yet another embodiment, treatment with the at least one active agent is maintained and treatment with Compound (I), or a pharmaceutically acceptable salt thereof, is reduced or discontinued (e.g., lower dose, less frequent dosing or shorter treatment regimen). In yet another embodiment, treatment with the at least one active agent and treatment with Compound (I), or a pharmaceutically acceptable salt thereof, is reduced or discontinued (e.g., lower dose, less frequent dosing or shorter treatment regimen).

Compound (I), or a pharmaceutically acceptable salt thereof, can be administered in combination with a MEK inhibitor selected from Trametinib, Cobimetinib, and Binimetinib. Accordingly, also provided is a method of treating cancer mediated by PKC comprising administering to a patient in need thereof, a therapeutically effective amount of 3-amino-N-(3-(4-amino-4-methylpiperidin-1-yl)pyridin-2-yl)-6-(3-(trifluoromethyl)pyridin-2-yl)pyrazine-2-carboxamide (Compound (I)), or a pharmaceutically acceptable salt thereof, in combination with a MEK inhibitor, in a dosing regimen comprising a first dosing cycle comprising a first dosing series followed by a second dosing series, wherein:

(a) the first dosing series comprises a dose of about 200 mg BID of compound (I) or a pharmaceutically acceptable salt thereof, and

(b) the second dosing series comprises a dose of about 400 mg BID of compound (I) or a pharmaceutically acceptable salt thereof.

### EXAMPLES

### Example 1

The following example presents a phase 1/2 study of Compound (I) in patients with solid tumors harboring GNAQ/11 mutations or PRKC fusions.

### Objectives

### Primary Outcome Measures:

Safety and identification of the maximum tolerated dose (MTD) and/or recommended phase 2 dose (RP2D), as assessed by the incidence of grade 3 or 4 adverse events (AEs) and clinically significant laboratory abnormalities defined as dose-limiting toxicities (DLTs). [Time Frame: 28 days]
Pharmacokinetic (PK) profile as assessed by the following PK parameters: [Time Frame: 28 days]
   Area under the curve (AUC) from Time zero to time t (AUC₀₋ₜ)
   AUC from time zero to infinity (AUC_{inf})
   AUC over the dosing interval (AUCₜₐᵤ)
   Maximum concentration (Cₘₐₓ)
   Time to maximum concentration (Tₘₐₓ)
   Elimination half-life (T½)
   Apparent volume of distribution at steady state after administration (Vₛₛ/F)
   Apparent total plasma clearance (CL/F)

### Secondary Outcome Measures

Anti-tumor activity in patients with metastatic uveal melanoma (MUM) as assessed by overall response rate (ORR) and duration of response (DOR) in accordance with the Response Evaluation Criteria in Solid Tumors (RECIST) Version 1.1 (v1.1) [Time Frame: until permanent treatment or study discontinuation]
Anti-tumor activity in patients with other tumor types harboring mutations in GNAQ or GNA11 as assessed by overall response rate (ORR) and duration of response (DOR) in accordance with the RECIST v1.1 [Time Frame: until permanent treatment or study discontinuation]
Comprehensive safety as assessed by the incidence of AEs or all grades and clinically significant laboratory abnormalities [Time Frame: until permanent treatment or study discontinuation]
Pharmacodynamic (PD) activity, assessed by the modulation of signaling proteins in the PKC pathway, e.g. PKC-delta [Time Frame: until permanent treatment or study discontinuation]

### Exploratory Outcome Measures

Anti-tumor activity in patients as assessed by progression-free survival (PFS) in accordance with RECIST v1.1 and overall survival (OS) [Time Frame: until permanent treatment or study discontinuation]
Correlation of tumor genetic and molecular characteristics and response to treatment, as assessed by gene signature profiling and/or molecular profiling, and objective response per RECIST v1.1 [Time Frame: until permanent treatment or study discontinuation]
Association of PD effects in tumor DNA and response, as assessed by cell-free or tumor tissue DNA and objective response per RECIST v1.1 [Time Frame: until permanent treatment or study discontinuation]

### Methodology:

This is a single-arm Phase 1/2 study. Therefore, no placebo or treatment arm was included.

### Experimental:

Patients with MUM were sequentially enrolled into Cohort 1 or Cohort 2. Enrollment into Cohort 2 was not initiated until enrollment into Cohort 1 was completed and the dose considered safe.
a. Cohort 1. 6 patients were sequentially enrolled and received 300 mg twice a day (BID), daily, for 28-day treatment cycles.
b. Cohort 2. 6 patients were sequentially enrolled and received 400 mg BID, preceded by an initial 7-day 200 mg BID run-in period, for 28-day treatment cycles.
   All patients continue to receive treatment with Compound (I) unless the subject experiences one or more of the following:
Adverse event
Lost to follow-up
Physician decision
Progressive disease
Study termination by the Sponsor
Patient/guardian decision
Protocol deviation
Non-compliance with protocol
Death
Pregnancy
   NUMBER OF SUBJECTS: 12 patients

### Criteria

### Inclusion Criteria:

Patient must be at least 18 years of age.
Patient is able to provide written, informed consent before initiation of any study-related procedures, and is able, in the opinion of the investigator, to comply with all the requirements of the study.
Patient Population
   Diagnosis of one of the following:
   Metastatic Uveal Melanoma (MUM): Uveal melanoma with histological or cytological confirmed metastatic disease. Metastatic disease may be treatment naive or have progressed (radiologically or clinically) on or after most recent therapy. Or
   Non-MUM: Advanced cutaneous melanoma, CRC, or other solid tumor that has either progressed following prior standard therapies or that has no satisfactory alternative therapies and has evidence of GNAQ/11 hotspot mutation (codons Q209 or R183) by local testing in a CAP/CLIA-certified laboratory.
   Representative archival metastatic tumor specimens in paraffin blocks with an associated pathology report or a minimum of 15 FFPE slides is mandatory. MUM patients who do not have archival tumor tissue samples at the time of study entry may still be eligible for the study.
Only tissue from a surgical resection or a core needle, punch, or excisional/incisional biopsy sample collection will be accepted. Fine needle aspiration (FNA) samples are not acceptable.
   Patients with tumors harboring non-hotspot GNAQ/11 mutations or PRKC fusions will be eligible for the Phase 2 expansion part of the study (future amendment).
Patients with a prior history of or clinically stable concurrent malignancy are eligible for enrollment provided the malignancy is clinically insignificant, no treatment is required, and the patient is clinically stable
   Patients with a history of squamous or basal cell carcinoma of the skin or carcinoma in the situ of the cervix may be enrolled.
   Patients with prostate cancer with an elevated PSA not requiring treatment may be enrolled
Measurable disease per RECIST v1.1, defined as at least one lesion that can be accurately measured in at least one dimension (longest diameter to be recorded) as ≥10 mm with CT or MRI scan, or by digital photography with calipers and ruler for cutaneous lesions. An enlarged lymph node must be ≥15 mm in short axis to be a measurable lesion.
   Lesions in previously irradiated areas should not be considered target lesions unless they have clearly progressed since the radiotherapy.
   Liver lesions that received liver-directed therapies should not be considered target lesions unless they have clearly progressed since the therapy.
   For patients with metastases in the liver, these patients should have contrast-enhanced liver imaging modality preference determined by expertise at the treating institution.
Patient has Eastern Cooperative Oncology Group (ECOG) performance status 0-1.
Patient has adequate organ function at screening:
   Absolute neutrophil count ≥1500/mm³ without the use of hematopoietic growth factors
   Platelet count ≥75,000/mm³ (must be at least 2 weeks post-platelet transfusion and not receiving platelet-stimulating agents)
   Hemoglobin ≥8.0 g/dL (must be at least 2 weeks post-red blood cell transfusion and not receiving erythropoietic-stimulating agents)
   Total bilirubin ≤1.5×the upper limit of normal (ULN). For patients with documented Gilbert's disease, total bilirubin ≤3.0 mg/dL is allowed
   Alanine aminotransferase (ALT) and aspartate aminotransferase (AST) ≤3×ULN in the absence of documented liver metastases; ≤5×ULN in the presence of liver metastases.
   Serum albumin ≥2.0 g/dL
   Creatinine Clearance ≥60 mL/min/1.73 m² by Cockroft-Gault equation
   Prothrombin time/International Normalized Ratio (INR) or partial thromboplastin time test results at screening ≤1.5×ULN (this applies only to patients who do not receive therapeutic anticoagulation; patients receiving therapeutic anticoagulation should be on a stable dose for at least 4 weeks prior to the first dose of study drug).
Patients who received prior immune-stimulatory antitumor agents, such as anti-PD-1, anti-PD-LI, anti-CTLA-4, OX-40, CD137, etc., or MAPK pathway inhibitors may be eligible. Prior to study Day 1 (first dose), patient must be:
   at least 4 weeks or 5 half-lives (T½) after the most recent biologic (antibody-based) or immunotherapy, whichever is shorter
   at least 2 weeks or 5 T½ after any prior chemotherapy (>6 weeks from nitrosourea and mitomycin C) or targeted small molecule therapy, whichever is shorter
Female patients of childbearing potential must be non-pregnant, non-lactating, and have a negative serum human chorionic gonadotropin pregnancy test result within 28 days prior to the first study drug administration.
   Females of childbearing potential who are sexually active with a non-sterilized male partner agree to use effective methods of contraception from screening, throughout the study drug and agree to continue using such precautions for 30 days after the final dose of study drug.
   Non-sterilized males who are sexually active with a female of childbearing potential must agree to use effective methods of contraception from Day 1 throughout the study drug and for 30 days after the final dose of study drug.

### Additional Inclusion Criteria for Non-Mum, Patients

Patients must have exhausted all standard treatments or have documented intolerance per the investigator.
Archival metastatic tumor specimens in paraffin blocks with an associated pathology report or a minimum of 15 FFPE slides is mandatory.
Only tissue from a surgical resection or a core needle, punch, or excisional/incisional biopsy sample collection will be accepted. Fine needle aspiration (FNA) samples are not acceptable.

### Cutaneous Melanoma

Histologically confirmed locally advanced and unresectable or metastatic melanoma with color medical grade photographs with a ruler if skin lesions present
Documented RAF/RAS wild-type status

### Colorectal Cancer:

Histologically confirmed locally advanced and unresectable or metastatic adenocarcinoma originating from the colon or the rectum
Documented RAF/RAS wild-type status and microsatellite stable (MSS) status

### Exclusion Criteria:

Previous treatment with a PKC inhibitor
Have AEs from prior anti-cancer therapy that have not resolved to Grade 51 except for alopecia, prior peripheral neuropathy, or anemia. Endocrinopathies resulting from previous immunotherapy are considered part of the medical history and not an AE.
Untreated or symptomatic central nervous system (CNS) metastases. Patients with asymptomatic CNS metastases are eligible provided they have been clinically stable and not requiring steroids for at least 4 weeks.
Known human immunodeficiency virus (HIV) or acquired immunodeficiency syndrome (AIDS)-related illness.
Known microsatellite instable-high (MSI-H) tumors will be excluded.
Active infection requiring therapy (except nail fungus), positive tests for Hepatitis B surface antigen (HBsAg) with detected Hepatitis B virus (HBV) DNA or positive Hepatitis C antibody with detected Hepatitis C virus (HCV) RNA.
Surgical procedures that require general anesthesia □5 days prior to first scheduled dose of Compound (I); in all cases, the patient must be sufficiently recovered and stable before study drug administration.
Prior gastrectomy or upper bowel removal or any other gastrointestinal disorder or defect e.g., malabsorption disorder such as Crohn's disease or ulcerative colitis, that would interfere with absorption of Compound (I).
Patients who received radiotherapy within 2 weeks of the first dose of study drug.
Patients who are receiving treatment with medications that cannot be discontinued prior to study entry and that are considered to be any of the following:
   known and possible risk for QT prolongation
   known to be strong inducers or inhibitors of CYP3A4/5
   known to be inducers or inhibitors of ABCB1 (P-gp)
   known to be substrates of CYP3A4/5, OAT3, OATP1B1, MATE1/2-K, and ABCB1 with a narrow therapeutic index
Females who are pregnant or breastfeeding:
   Women of childbearing potential must not be considering getting pregnant during the study.
   Patients of reproductive potential (male & female) must practice an effective method of contraception during treatment and for 30 days following the last dose of Compound (I). Patients unwilling to do so will be excluded.
Impaired cardiac function or clinically significant cardiac diseases, including any of the following:
   History or presence of ventricular tachyarrhythmia
   Presence of unstable atrial fibrillation (ventricular response >100 BPM); patients with stable atrial fibrillation are eligible, provided they do not meet any of the other cardiac exclusion criteria
   Angina pectoris or acute myocardial infarction ≤6 months prior to starting study drug
   Other clinically significant heart disease (e.g., symptomatic congestive heart failure; uncontrolled arrhythmia or hypertension; history of labile hypertension or poor compliance with an antihypertensive regimen)
   Patients with a drug eluting stent for cardiovascular purposes placed ≤6 months prior to starting study drug
   Corrected QT interval using Fridericia's method (QTcF)>480 msec on baseline ECG (mean of baseline values). If electrolytes are abnormal, they may be corrected, and baseline ECGs should be repeated
For subjects receiving Compound (I) Powder-In-Capsule only: allergy to mammalian meat products or gelatin.
Presence of any other condition that may increase the risk associated with study participation or may interfere with the interpretation of study results and, in the opinion of the investigator, would make the patient inappropriate for entry into the study.

### Example 2

The following example presents pharmacokinetic results from the study as described in Example 1.

Blood samples were collected on Cycle 1 Day 1 and Day 15 from patients who were administered 300 mg BID of Compound (I), and on Cycle 1 Day 8 and Day 22 from patients who were administered 200 mg BID for 7 days followed by 400 mg BID of Compound (I) for the remainder of the treatment cycle. The plasma samples were processed by protein precipitation (see Ahuja S and Dong M. W, Handbook of Pharmaceutical Analysis by HPLC, Elsevier Inc., Chapter 17, page 433, 2005) and analyzed using an LC-MS/MS method; Quantification was achieved using Compound (I) peak area to internal standard (3-amino-N-(3-(4-amino-4-(methyl-d3)piperidin-1-yl)pyridin-2-yl)-6-(3-(trifluoromethyl)pyridin-2-yl)pyrazine-2-carboxamide) peak area ratios. Concentrations of the calibration curve standards, quality control samples, and patient samples were determined by the method of 1/x² weighted least squares linear regression.

The Pharmacokinetic parameters were calculated using Phoenix WinNonlin software (non-compartmental analysis).

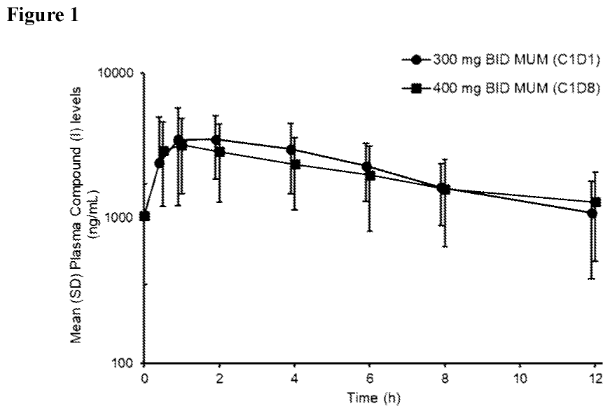

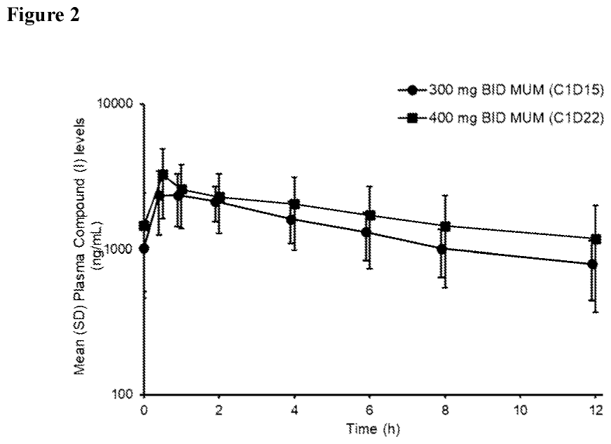

FIGS. 1 and 2 illustrate plasma concentrations vs. time following a single dose (C1D1 for 300 mg and C1D8 for 400 mg) and at steady state (C1D15 for 300 mg and C1D22 for 400 mg), respectively.

FIG. 1 shows the mean (SD) plasma concentration vs. time following a single dosing of Compound (I) at 300 mg BID on C1D1 (n=12) and 400 mg BID with 200 mg run-in on C1D8 (n=24).

FIG. 2 shows the mean (SD) plasma concentration vs. time following a single dosing of Compound (I) at 300 mg BID on C1D15 (n=12) and 400 mg BID with 200 mg run-in on C1D22 (n=21).

The pharmacokinetic parameters are listed in Tables 1-3.

TABLE 1
Pharmacokinetic parameters of Compound (I) following single or repeat
dosing at 300 mg BID or 400 mg BID with 200 mg run-in (Mean ± SD)

| | | | | | Cₘᵢₙ | |
|---|---|---|---|---|---|---|
| Dose | Cₘₐₓ (ng/mL) | | AUC₀₋₁₂ (ng · h/mL) | | (ng/mL) | |
| (mg) | D1/D8 | D15/D22 | D1/D8 | D15/D22 | D15/D22 | R_{ace} |
| 300 BID | 4598 ± 2297 | 2856 ± 690 | 26159 ± 11058 | 16827 ± 5379 | 790 ± 345 | 0.74 ± 0.31 |
| (n = 12) | | | | | | |
| 400 BID | 3934 ± 1882 | 3582 ± 1389 | 24406 ± 12127 | 21507 ± 10837 | 1120 ± 806 | 0.97 ± 0.29 |
| with run-in | | | | | | |
| (n = 24) | | | | | | |

At steady state, the mean Cₘᵢₙ at 400 mg BID (with 200 mg run-in) was ˜40% higher than that at 300 mg BID.

TABLE 2
Individual patient pharmacokinetic parameters of Compound (I)
following single or repeat dosing at 300 mg BID

| 300 mg | | | | | AUC₀₋₁₂ | | Cₘᵢₙ on | AUC |
|---|---|---|---|---|---|---|---|---|
| BID | Cₘₐₓ (ng/mL) | | Tₘₐₓ (h) | | (ng · h/mL) | | C1D15 | ratio |
| C1 Day | D1 | D15 | D1 | D15 | D1 | D15 | (ng/mL) | D15/D1 |
| 10001- | 2256 | 2971 | 2 | 0.5 | 19768 | 13083 | 699 | 0.66 |
| 11001 | | | | | | | | |
| 10001- | 3159 | 3965 | 4 | 0.5 | 22974 | 19049 | 645 | 0.83 |
| 11002 | | | | | | | | |
| 10001- | 2980 | 2149 | 2 | 1 | 17983 | 15640 | 626 | 0.87 |
| 11003 | | | | | | | | |
| 10001- | 607 | 1392 | 1 | 2 | 3597 | 5511 | 175 | 1.53 |
| 11004\hat{ } | | | | | | | | |
| 10004- | 3147 | 3079 | 2 | 0.5 | 22480 | 15071 | 792 | 0.67 |
| 11005 | | | | | | | | |
| 10003- | 4367 | 2984 | 1 | 1 | 19439 | 21194 | 1075 | 1.1 |
| 11006 | | | | | | | | |
| 10001- | 8940 | 3442 | 1 | 1 | 35890^{a} | 23600 | 1212 | 0.66 |
| 00022 | | | | | | | | |
| 10001- | 7009 | 2782 | 0.5 | 0.5 | 43190 | 19185 | 1088 | 0.44 |
| 00026 | | | | | | | | |
| 10003- | 5386 | 2574 | 2 | 1 | 35321 | 17909 | 798 | 0.51 |
| 00019 | | | | | | | | |
| 10003- | 6252 | 2969 | 0.5 | 0.5 | 26969 | 10949 | 322 | 0.41 |
| 00020 | | | | | | | | |
| 10004- | 5549 | 3618 | 2 | 1 | 39949 | 24582 | 1337 | 0.62 |
| 00013 | | | | | | | | |
| 10002- | 3866 | 4203 | 1 | 0.5 | 233274 | 28493 | 1549 | 1.22 |
| 00014* | | | | | | | | |
| 10003- | 5523 | 2348 | 0.5 | 1 | 26345 | 16155 | 715 | 0.61 |
| 00027 | | | | | | | | |

^{a}AUC₀₋₈
*non-MUM patient
\hat{ }Patient was on herbal supplement that potentially contained CYP3A4 inducer

TABLE 3
Individual patient pharmacokinetic parameters of Compound (I)
following single or repeat dosing at 400 mg BID with 200 mg run-in

| 400 mg | | | | | AUC₀₋₁₂ | | Cₘᵢₙ on | AUC |
|---|---|---|---|---|---|---|---|---|
| BID | Cₘₐₓ (ng/mL) | | Tₘₐₓ (h) | | (ng · h/mL) | | C1D22 | ratio |
| C1 Day | D8 | D22 | D8 | D22 | D8 | D22 | (ng/mL) | D22/D8 |
| 10001- | 3053 | 3890 | 0.5 | 1 | 13604 | 18178 | 677 | 1.3 |
| 21001 | | | | | | | | |
| 10001- | 2068 | 2556 | 2 | 2 | 17779 | 22282 | 1019 | 1.3 |
| 21002 | | | | | | | | |
| 10004- | 2948 | 2756 | 1 | 1 | 19720 | 21010 | 969 | 1.1 |
| 21003 | | | | | | | | |
| 10001- | 2077 | 1709 | 6 | 6 | 22367 | 18847 | 931 | 0.84 |
| 00011 | | | | | | | | |
| 10001 | 7600 | 5026 | 0.5 | 0.5 | 23416 | 15424 | 497 | 0.66 |
| 00015 | | | | | | | | |
| 10004- | 2121 | 2562 | 1 | 0.5 | 16365 | 12278 | 533 | 0.75 |
| 00012 | | | | | | | | |
| 10001- | 2580 | NA | 2 | NA | 28796 | NA | NA | NA |
| 00021 | | | | | | | | |
| 10001- | 1949 | 1911 | 1 | 1 | 13072 | 13221 | 674 | 1.0 |
| 00028 | | | | | | | | |
| 10001- | 7450 | NA | 1 | NA | 56156 | NA | NA | NA |
| 00033 | | | | | | | | |
| 10004- | 3838 | 2853 | 0.5 | 0.5 | 22089 | 18705 | 916 | 0.85 |
| 00025 | | | | | | | | |
| 10004- | 6560 | NA | 0.5 | NA | 23178 | NA | NA | NA |
| 00032 | | | | | | | | |
| 10001- | 4478 | 3934 | 1 | 0.5 | 18745 | 20068 | 777 | 1.1 |
| 00034 | | | | | | | | |
| 10002- | 1922 | 2578 | 0.5 | 0.5 | 6311 | 6604 | 179 | 1.0 |
| 00017 | | | | | | | | |
| 10002- | 6872 | 5948 | 1 | 0.5 | 38119 | 36219 | 1631 | 0.95 |
| 00024 | | | | | | | | |
| 10002- | 4037 | 2203 | 0.5 | 0.5 | 13309 | 7788 | 334 | 0.59 |
| 00031 | | | | | | | | |
| 10002- | 2949 | 2403 | 2 | 0.5 | 22361 | 19634 | 1110 | 0.88 |
| 00044 | | | | | | | | |
| 10003- | 2603 | 1992 | 0.5 | 1 | 10348 | 10656 | 264 | 1.0 |
| 00029 | | | | | | | | |
| 10004- | 3474 | 5663 | 0.5 | 0.5 | 32689 | 48381 | 3140 | 1.5 |
| 00038 | | | | | | | | |
| 10004- | 7043 | 5314 | 2 | 0.5 | 48672 | 20635 | 1082 | 0.42 |
| 00039 | | | | | | | | |
| 10004- | 2520 | 3276 | 4 | 1 | 23856 | 28583 | 1887 | 1.2 |
| 00042 | | | | | | | | |
| 10004- | 2531 | 4296 | 1 | 0.5 | 17650 | 11327 | 478 | 0.64 |
| 00043 | | | | | | | | |
| 11001- | 4952 | 5657 | 2 | 0.5 | 43163 | 30027 | 1912 | 0.70 |
| 00046 | | | | | | | | |
| 10002- | 4452 | 5617 | 0.5 | 0.5 | 23378 | 32888 | 1592 | 1.4 |
| 00040 | | | | | | | | |
| 10002- | 4329 | 3676 | 2 | 4 | 30601 | 38889 | 2909 | 1.3 |
| 00045 | | | | | | | | |

The mean free plasma AUC₀₋₁₂ at 400 mg, BID of Compound (I) with a 200 mg run-in was about 44% higher than that at 300 mg BID of Compound (I). This indicates that a dosing regimen of 400 mg BID (after a 200 mg, BID run-in) results in a higher average steady state exposure of free Compound (I) in comparison to a 300 mg BID dosing regimen as exemplified in Table 4 below.

TABLE 4
Mean free plasma AUC₀₋₁₂ of
Compound (I) at 400 mg BID
(with 200 mg run-in) compared to 300 mg BID

| | Free AUC₀₋₁₂ |
|---|---|
| Dose | (ng · h/mL) |
| 300 mg | 260 ± 95 |
| 400 mg with | 396 ± 222 |
| 200 mg run-in | |

Based on the current data, patients who were administered 200 mg BID for first 7 days, followed by 400 mg BID of Compound (I) had lower mean exposure on C1D8 compared to C1D1 of patients who received 300 mg BID. Additionally, patients who were administered 200 mg BID for first 7 days, followed by 400 mg BID of Compound (I) had higher mean steady state exposure of Compound (I) vis-A-vis patients who were administered 300 BID (RDE) of Compound (I) for the same duration.

### Hypotension Studies

TABLE 5
All adverse events occurring in > 10% or grade 3/4
adverse events occurring in > 5% of all
patients treated on single agent Compound
(I) BID schedule, regardless of study drug
relationship by preferred term and treatment dose

| | 300 mg BID | | 400 mg | | All BID Patients | |
|---|---|---|---|---|---|---|
| Compound (I) | N = 18 | | N = 6 | | N = 30 | |
| Grade | All | >3 | All | >3 | All | >3 |
| Hypotension | 3 (16.7) | 0 | 4 (66.7) | 1 (16.7) | 7 (23.3) | 1 (3.3) |

TABLE 6
All and grade 3/4 adverse events occurring in > 5% of all patients,
regardless of study drug relationship by preferred term and treatment
schedule-single-agent Compound (I)

| | 300 mg BID | | 400 mg BID* | | All Patients | |
|---|---|---|---|---|---|---|
| Preferred | (N = 26) | | (N = 30) | | (N = 56) | |
| Term | All | 3/4 | All | 3/4 | All | 3/4 |
| Grade | n (%) | n (%) | n (%) | n (%) | n (%) | n (%) |
| Hypotension | 1 (3.8) | 0 | 4 (13.3) | 0 | 5 (8.9) | 0 |

*the 400 mg BID dose cohort has a 7-day 200 mg run-in period

TABLE 7
Treatment-emergent Adverse Events Occurring in > 2% Patients
Suspected to be Study Related by Preferred Term
Safety Population-single agent Compound (I)

| | 300 mg BID | | 400 mg BID* | | All Patients | |
|---|---|---|---|---|---|---|
| Preferred | (N = 26) | | (N = 30) | | (N = 56) | |
| Term | All | 3/4 | All | 3/4 | All | 3/4 |
| Grade | n (%) | n (%) | n (%) | n (%) | n (%) | n (%) |
| Hypotension | 1 (3.8) | 0 | 2 (6.7) | 0 | 3 (5.4) | 0 |

*the 400 mg BID dose cohort has a 7-day 200 mg run-in period

As can be seen in the tables above, hypotension occurs less frequently when Compound (I) is administered to patients at 400 mg BID with a 200 mg run-in than 400 mg BID alone.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A method of treating cancer mediated by protein kinase C comprising administering to a patient in need thereof, a therapeutically effective amount of 3-amino-N-(3-(4-amino-4-methylpiperidin-1-yl)pyridin-2-yl)-6-(3-(trifluoromethyl)pyridin-2-yl)pyrazine-2-carboxamide (Compound (I)), or a pharmaceutically acceptable salt thereof, as a monotherapy, in a dosing regimen comprising a first dosing cycle comprising a first dosing series, followed by a second dosing series, wherein:
(a) the first dosing series comprises a dose of about 200 mg BID of compound (I), or a pharmaceutically acceptable salt thereof, and
(b) the second dosing series comprises a dose of about 400 mg BID of compound (I), or a pharmaceutically acceptable salt thereof.

2. The method of claim 1, wherein the length of the first dosing series is 5 to 10 days.

3. The method of claim 1, wherein the length of the second dosing series is 18 to 23 days; provided the length of first dosing cycle comprising first dosing series and second dosing series is 28 days.

4. The method of claim 2, wherein the first dosing cycle comprises one first dosing series, and compound (I) is administered on days 1 to 5 consecutively, of the first dosing series of the first dosing cycle.

5. The method of claim 2, wherein the first dosing cycle comprises one first dosing series, and compound (I) is administered on days 1 to 6 consecutively, of the first dosing series of the first dosing cycle.

6. The method of claim 2, wherein the first dosing cycle comprises one first dosing series, and compound (I) is administered on days 1 to 7 consecutively, of the first dosing series of the first dosing cycle.

7. The method of claim 4, wherein the first dosing cycle comprises one second dosing series, and compound (I) is administered on days 6 to 28 consecutively, of the first dosing cycle or days 1 to 23 of the second dosing series.

8. The method of claim 5, wherein the first dosing cycle comprises one second dosing series, and compound (I) is administered on days 7 to 28 consecutively, of the first dosing cycle or days 1 to 22 of the second dosing series.

9. The method of claim 6, wherein the first dosing cycle comprises one second dosing series, and compound (I) is administered on days 8 to 28 consecutively, of the first dosing cycle or days 1 to 21 of the second dosing series.

10. The method of claim 1, wherein the dosing regimen comprises one or more additional dosing cycles of second dosing series wherein each additional dosing cycle is 28 days.

11. The method of claim 10, wherein compound (I) is administered consecutively for 28 days of each additional dosing cycle.

12. The method of claim 10 wherein the number of additional dosing cycles of second dosing series is at least 4.

13. The method of claim 10 wherein the number of additional dosing cycles of second dosing series is at least 8.

14. The method of claim 10 wherein the number of additional dosing cycles of second dosing series is at least 10.

15. The method of claim 10 wherein the number of additional dosing cycles of second dosing series is at least 12.

16. The method of claim 10 wherein the number of additional dosing cycles of second dosing series is at least 16.

17. The method of claim 10 wherein the number of additional dosing cycles of second dosing series is at least 24.

18. The method of claim 1, wherein the patient is administered 200 mg BID of compound (I) in the first dosing series and 400 mg BID of compound (I) in each of the second dosing series.

19. The method of claim 1, wherein the cancer is selected from the group consisting of melanoma, uveal melanoma, lymphoma, diffuse large B-cell lymphoma (DLBCL), ibrutinib resistant cancers, pancreatic cancer, colorectal cancer, lung adenocarcinoma, cutaneous melanoma, stomach cancer, cervical cancer, uterine cancer, bladder cancer, hepatocellular carcinoma, prostate cancer, breast cancer, head and neck cancer, and glioblastoma.

20. The method of claim 19, wherein the cancer is uveal melanoma.

21. The method of claim 20, wherein the cancer is metastatic uveal melanoma

22. The method of claim 19, wherein the cancer is selected from the group consisting of pancreatic cancer, colorectal cancer, lung adenocarcinoma, cutaneous melanoma, stomach cancer, cervical cancer, uterine cancer, bladder cancer, hepatocellular carcinoma, prostate cancer, breast cancer, head and neck cancer, and glioblastoma.

23. The method of claim 20, wherein the patient being treated harbors GNAQ or GNA11 mutations.

24. The method of claim 19, wherein the cancer is diffuse large B-cell lymphoma (DLBCL).

25. The method of claim 24, wherein diffuse large B-cell lymphoma (DLBCL) is treated in a patient harboring CD79 mutations.
